(19) United States
(12) Patent Application Publication
(10) Pub. No.: US 2021/0204855 A1
(43) Pub. Date: Jul. 8, 2021

(54) BIOSIGNAL MEASURING AND STIMULATING DEVICE HAVING BIOELECTRODE

(71) Applicant: INDUSTRY-ACADEMIC COOPERATION FOUNDATION, YONSEI UNIVERSITY, Seoul (KR)

(72) Inventors: Heon-Jin CHOI, Seoul (KR); Youngcheol CHAE, Seoul (KR); Jae-Suk SUNG, Suwon-Si (KR); Ju Kwan NA, Seoul (KR)

(21) Appl. No.: 17/272,754

(22) PCT Filed: Sep. 2, 2019
(86) PCT No.: PCT/KR2019/011261

§ 371 Date: Mar. 2, 2021

(30) Foreign Application Priority Data
Sep. 4, 2018 (KR) 10-2018-0105113
Aug. 5, 2019 (KR) 10-2019-0094932

(51) Int. Cl. A61B 5/25; A61B 5/00; A61N 1/04

(52) U.S. Cl. CPC A61B 5/25; A61B 5/002; A61N 1/0476; A61B 2560/0214; A61B 2562/0209; A61B 2562/046; A61B 2562/028; A61B 2562/125

(57) ABSTRACT

The present invention provides a biosignal measuring and stimulating device having bioelectrodes in which a signal measurement unit (200) including bioelectrodes composed of a plurality of microelectrodes (210) is disposed on a substrate (100), wherein the signal measurement unit (200), a measured signal processing unit (300), and at least one of a driving power unit (400) and a wireless communication unit (500) are disposed on the substrate (100) in a vertical or lateral direction, and the biosignal measuring and stimulating device measures biosignals or stimulates from the microelectrodes formed in an array pattern.

### TECHNICAL FIELD

The present invention relates to a biosignal measuring and stimulating device. More particularly, the present invention relates to a biosignal measuring and stimulating device having bioelectrodes.

### BACKGROUND ART

The present invention is directed to a technology for measuring neural biosignals, analyzing the signals, and then applying appropriate stimuli, and more specifically to a biosignal measuring and stimulating structure for accurately measuring and analyzing biosignals in order to inquire into the biological structure/function of a living organism such as a human body, an animal, or a plant.

Various methods for precisely measuring such neural biosignals are being researched. In addition, research is being conducted on a method of reducing pain and treating a nerve-related problem by applying predetermined stimuli around a nerve.

However, in connection with the measurement of biosignals, there are problems in that it is impossible to simultaneously measure a large number of biosignals by using the prior art and there are many limitations in terms of the resolution of signal measurement.

Furthermore, there is a problem in that a long manufacturing time is required because it is difficult to perform the process of coupling separate microelectrodes to a substrate through soldering or the like.

### DISCLOSURE

### Technical Problem

A biosignal measuring and stimulating device having bioelectrodes according to the present invention has the following objects:

First, the present invention is intended to arrange components so that a biosignal measuring device can be reduced in size.

Second, the present invention is intended to simultaneously secure a plurality of measured values of biosignals.

Third, the present invention is intended to wirelessly transmit measured values by using wireless communication technology.

Fourth, the present invention is intended to simplify the process of manufacturing microelectrodes.

Fifth, the present invention is intended to overcome the limitation of the resolution aspect of biosignals.

The objects of the present invention are not limited to those described above, and other objects that are not described will be clearly understood by those skilled in the art from the following description.

### Technical Solution

The present invention provides a biosignal measuring and stimulating device having bioelectrodes in which a signal measurement unit including bioelectrodes composed of a plurality of microelectrodes is disposed on a substrate, wherein the signal measurement unit, a measured signal processing unit, and at least one of a driving power unit and a wireless communication unit are disposed on the substrate in a vertical or lateral direction, and the biosignal measuring and stimulating device measures biosignals or stimulates from the microelectrodes formed in an array pattern.

The microelectrodes according to the present invention may be provided as solder bumps.

The solder bumps according to the present invention may have a round shape or a tapered cone shape widening downward.

The driving power unit according to the present invention may be any one of a coin-type battery, a film-type thin film battery, a piezoelectric-type rechargeable battery, a triboelectric-type rechargeable battery, a solar-type wireless power transmission unit, an RF wireless power transmission unit, a biofuel cell, and a super-capacitor.

The wireless communication unit according to the present invention may use any one of a Bluetooth communication device, a Wi-Fi communication device, and a BCC communication device.

In the present invention, the microelectrodes formed in the array pattern may be set as one microelectrode, which is a reference electrode, and a plurality of corresponding electrode groups each including other microelectrodes located at the same distance from the reference electrode, and, for each of the corresponding electrode groups, the average of the measured values of biosignals between the reference electrode and the micro-electrodes of the corresponding electrode group may be obtained.

In the present invention, the average of measured values obtained by excluding at least one of an upper limit value and a lower limit value from measured values for each of the corresponding electrode groups may be obtained.

Preferably, the plurality of microelectrodes according to the present invention are arranged in a pattern where the same numbers of microelectrodes are arranged in the lateral and transverse directions of the pattern, and a reference electrode is any one of the plurality of microelectrodes.

The plurality of microelectrodes according to the present invention may be arranged such that the same odd numbers of microelectrodes are arranged in the lateral and transverse directions of the pattern, and the reference electrode may be set to a microelectrode located at the center of the pattern.

Preferably, the plurality of microelectrodes according to the present invention are arranged in a pattern where different numbers of microelectrodes are arranged in the lateral and transverse directions of the pattern, and a reference electrode is any one of the plurality of microelectrodes.

Preferably, the plurality of microelectrodes according to the present invention are disposed at the center of a plurality of concentric circles and on the circumferences of the respective concentric circles, a reference electrode is a microelectrode disposed at the center of the circles, and each corresponding electrode group includes microelectrodes arranged on the circumference of a corresponding one of the concentric circles.

### Advantageous Effects

The biosignal measuring and stimulating device having bioelectrodes according to the present invention has the following effects:

First, the present invention has the effect of reducing the biosignal measuring device to a small size by arranging the signal measurement unit, the measured signal processing unit, the driving power unit, and the wireless communication unit in a vertical or transverse direction.

Second, the present invention has the effect of utilizing the microelectrode array pattern structure, thereby securing a large number of high-precision measured values and significantly reducing measurement cost and measurement time.

Third, the present invention has the effect of easily and wirelessly transmitting measured values by using a wireless communication device such as a Bluetooth communication device, a Wi-Fi communication device, or a BCC communication device.

Fourth, the present invention has the effect of using solder bumps as microelectrodes, thereby simplifying the process of manufacturing the microelectrodes.

The effects of the present invention are not limited to those described above, and other effects that are not described will be clearly understood by those skilled in the art from the following description.

### DESCRIPTION OF DRAWINGS

FIGS. 1a to 1c are schematic diagrams showing various embodiments of the structure of a biosignal measuring device according to the present invention;

FIGS. 2a to 2c are schematic diagrams showing the overall shape and vertical section of a biosignal measuring device according to the present invention;

FIGS. 3a and 3b show embodiments of solder bumps according to the present invention;

FIG. 4 shows an embodiment in which the components of a biosignal measuring device according to the present invention are arranged in a lateral direction; and

FIGS. 5 to 7 show various embodiments of a microelectrode array pattern according to the present invention.

### BEST MODE

The present invention provides a biosignal measuring and stimulating device having bioelectrodes in which a signal measurement unit including bioelectrodes composed of a plurality of microelectrodes is disposed on a substrate, wherein the signal measurement unit, a measured signal processing unit, and at least one of a driving power unit and a wireless communication unit are disposed on the substrate in a vertical or lateral direction, and the biosignal measuring and stimulating device measures biosignals or stimulates from the microelectrodes formed in an array pattern.

### MODE FOR INVENTION

Embodiments of the present invention will be described with reference to the accompanying drawings below so that those of ordinary skill in the art to which the present invention pertains can easily practice the present invention. As can be easily understood by those of ordinary skill in the art to which the present invention pertains, the embodiments to be described later may be modified in various forms without departing from the spirit and scope of the present invention. The same or similar parts are denoted by the same reference numerals throughout the drawings as much as possible.

The technical terms used in this specification are intended to refer only to specific embodiments, but are not intended to limit the invention. As used herein, singular forms also include plural forms unless the phrases clearly indicate the opposite.

The meaning of “including” used herein specifies specific features, regions, integers, steps, acts, elements, and/or components, but does not exclude the presence or addition of another specific feature, region, integer, step, act, element, component, and/or group.

All terms including technical and scientific terms used herein have the same meanings as commonly understood by those of ordinary skill in the art to which the present invention pertains. The terms defined in dictionaries are further interpreted as having meanings consistent with related technical literature and the presently disclosed content, and are not interpreted as having ideal or excessively formal meanings unless defined as such.

The present invention will be described with reference to the drawings below. For reference, the drawings may be partially exaggerated to describe the features of the present invention. In this case, it is preferable to interpret the drawings in light of the overall purpose of the present specification.

The present invention is characterized by a biosignal measurement device in which a signal measurement unit 200 including biological electrodes composed of a plurality of microelectrodes 210 is disposed on the substrate 100, wherein the signal measurement unit 200, a measured signal processing unit 300, and at least one of a driving power unit 400 and a wireless communication unit 500 are disposed on the substrate 100 in a vertical or lateral direction and the biosignal measurement device measures biosignals or stimulates from the microelectrodes formed in an array pattern.

The present invention is directed to a device for measuring biosignals and stimulating when necessary, and more specifically to the basic mechanism and structure of a device for ultra-precise, high-resolution measurement/analysis required for the inquiry into a biological structure and function.

In general, in order to measure biosignals or stimulate, a ‘signal measurement unit’ for measuring biosignals and a ‘measured signal processing unit’ for processing a large number of measured signals are required.

Furthermore, a ‘driving power unit’ for driving the above devices may be included, and a ‘wireless communication unit’ for transmitting obtained signals to the outside may be included.

In other words, there is the ‘signal measurement unit’ as the most core structure for measuring biosignals, and an overall module is formed in a structure additionally including the ‘measured signal processing unit,’ the ‘driving power unit,’ and the ‘wireless communication unit.’

Furthermore, both a flexible PCB substrate form and a rigid PCB substrate form may be applied as the substrate according to the present invention. However, in order to increase the contact property of a biosensor, it will be preferable to use a flexible substrate.

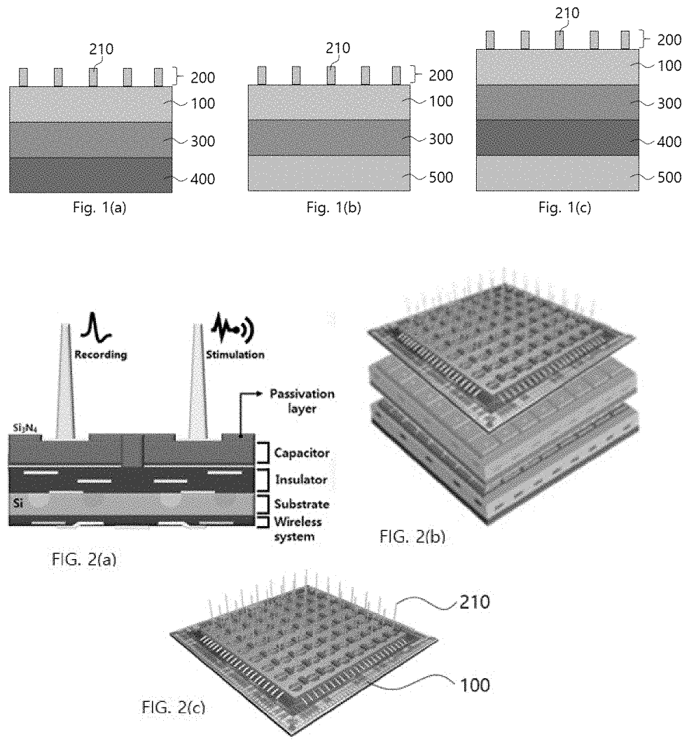

FIG. 1 is a schematic diagram showing various embodiments of the structure of a biosignal measuring device according to the present invention, in each of which components according to the present invention are arranged in a vertical direction.

The biosignal measuring device according to the present invention may include a substrate 100, a signal measurement unit 200, and a measured signal processing unit 300, and may selectively include a driving power unit 400 and a wireless communication unit 500.

FIGS. 1a to 1c are schematic diagrams showing the structures of a biological signal measuring device according to the present invention. As described above, a ‘neural signal measurement unit’ for reading biosignals or stimulating may be located on a top end surface, and may be connected to a ‘measured signal processing unit’ for processing biosignals.

For example, a ‘driving power unit’ may be configured not to be external but to be integrated into a module and to supply power, as shown in FIG. 1a, a ‘wireless communication unit’ for transmitting and receiving measured signals may be integrated into a module, as shown in FIG. 1b, and both a ‘driving power unit’ and a ‘wireless communication unit’ may be integrated into a module, as shown in FIG. 1c.

More specifically, the embodiment of FIG. 1a is an embodiment in which a driving power unit 400-a measured signal processing unit 300-a substrate 100-a signal measurement unit 200 are provided in the vertical direction thereof from the bottom thereof.

The embodiment of FIG. 1b is an embodiment in which a wireless communication unit 500-a measured signal processing unit 300-a substrate 100-a signal measurement unit 200 are provided in the vertical direction thereof from the bottom thereof.

The embodiment of FIG. 1c is an embodiment in which a wireless communication unit 500-a driving power unit 400-a measured signal processing unit 300-a substrate 100-a signal measurement unit 200 are provided in the vertical direction thereof from the bottom thereof.

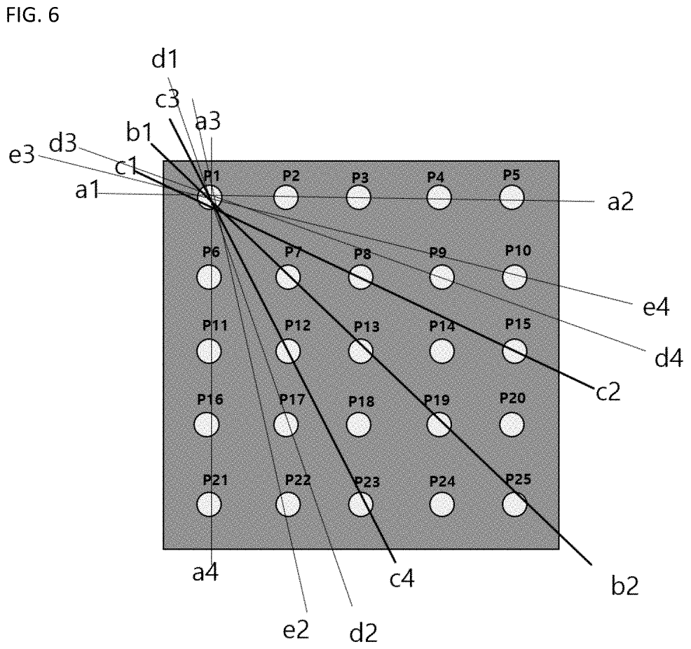

Meanwhile, although the individual technical components are arranged in the vertical direction in the embodiments of FIGS. 1a to 1c, there may be possible an embodiment in which components are arranged in the lateral direction thereof on a horizontal plane, as shown in FIG. 4.

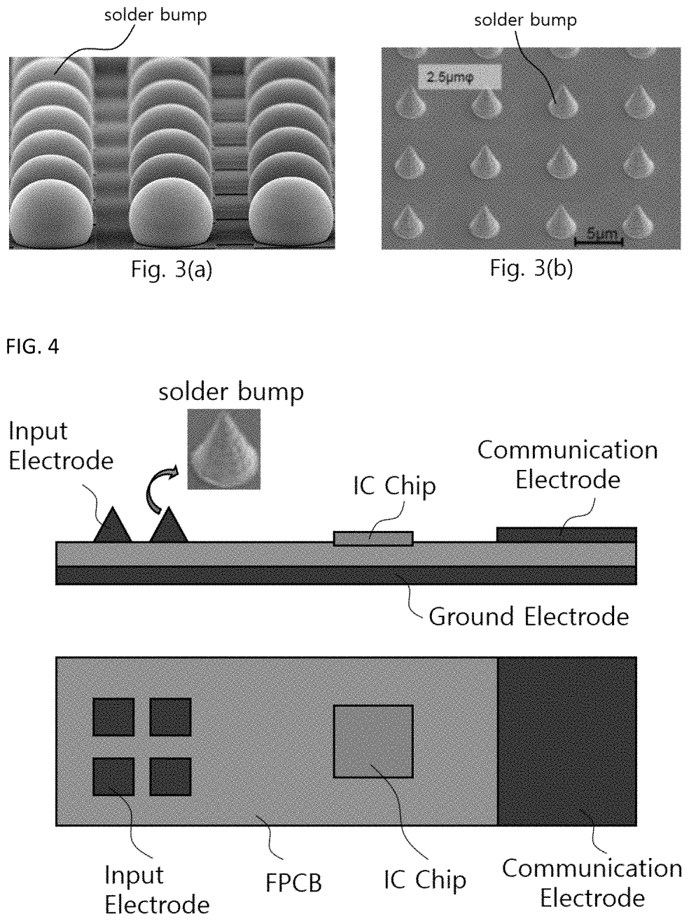

FIGS. 2a to 2c are schematic diagrams showing the overall shape and vertical section of a biosignal measuring device according to the present invention. As shown in FIGS. 2a to 2c, the electrodes of the ‘neural signal measurement unit’ not only act to measure biosignals, but also act as stimuli adapted to apply constant current to a nerve.

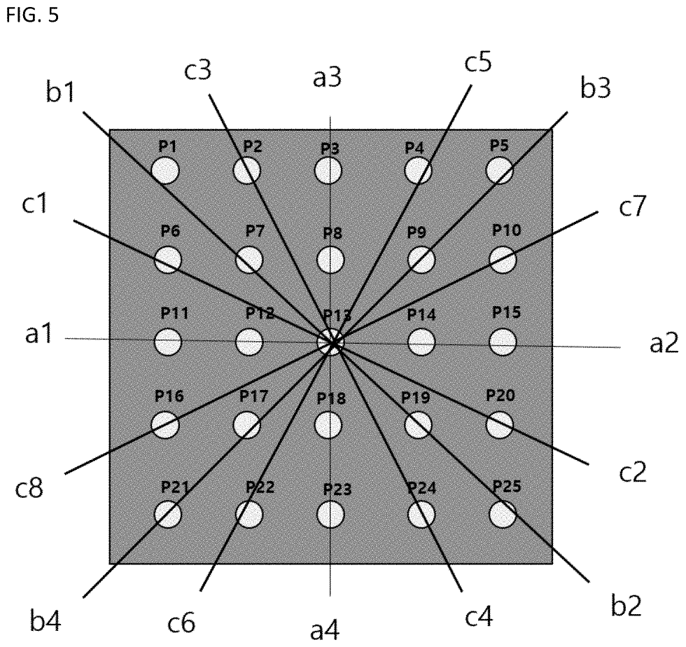

In the present invention, it is preferable that the microelectrodes 210 of the signal measurement unit 200 may be provided in the form of solder bumps SB and the solder bumps SB may be provided in a round shape or a cone shape tapering upward (see FIGS. 3 and 4).

In the present invention, it may also be possible to use existing commercial electrodes. Furthermore, in order to increase a property of contact with a living body, solder bumps may be formed on electrode portions on the substrate 100, and thus the solder bumps themselves may be implemented as a 3D microelectrode structure.

General solder bumps are used for connection between circuits of a substrate, and commonly have a round ball shape. The size of the ball shape is about 100-200 μm. In the case of micro-bumps, the size is reduced to about 15-30 μm. With the development of such miniaturization, micro-bumps in a cone shape have recently emerged. The diameter of the cone bumps is a minimum of 2.5 μm. These cone bumps are also used for connection between circuits while being soldered.

The present invention is characterized in that the micro-bumps themselves in a ball shape (a round shape) or a cone shape (a tapered shape) are utilized as 3D microelectrodes. In other words, for example, cone-shaped bumps are surface-mounted (SMT) on electrodes of a PCB substrate, and the surface-mounted bumps themselves are used as bioelectrodes without an additional soldering process.

In this case, compared to the conventional 2D electrodes of the PCB board, the property of contact with a living body may be significantly increased, so that contact impedance may be lowered, which makes it possible to monitor biosignals desirably and also facilitates the role of current stimulation.

Meanwhile, when solder bumps are used as bioelectrodes as suggested by the present invention, an electrode formation process may be facilitated and the contact property may be improved.

In the present invention, the bioelectrodes are implemented in an array electrode form. Through this, an advantage arises in that a considerably large amount of data may be obtained at one time. When an average is taken from a large amount of data, it may be possible to achieve high reliability.

In the present invention, the microelectrodes formed in an array pattern may be set as one microelectrode, which is a reference electrode, and a plurality of corresponding electrode groups each including other microelectrodes located at the same distance from the reference electrode. For each of the corresponding electrode groups, the average of the measured values of biosignals between the reference electrode and the micro-electrodes of the corresponding electrode group may be obtained.

In the present invention, the biosignals refer to signals obtained by measuring a phenomenon of a human body in an invasive or non-invasive manner. For example, the biosignals include various biosignals such as electrocardiogram signals, electroencephalogram signals, and electromyography signals, and may be measured in various forms such as capacitance and impedance.

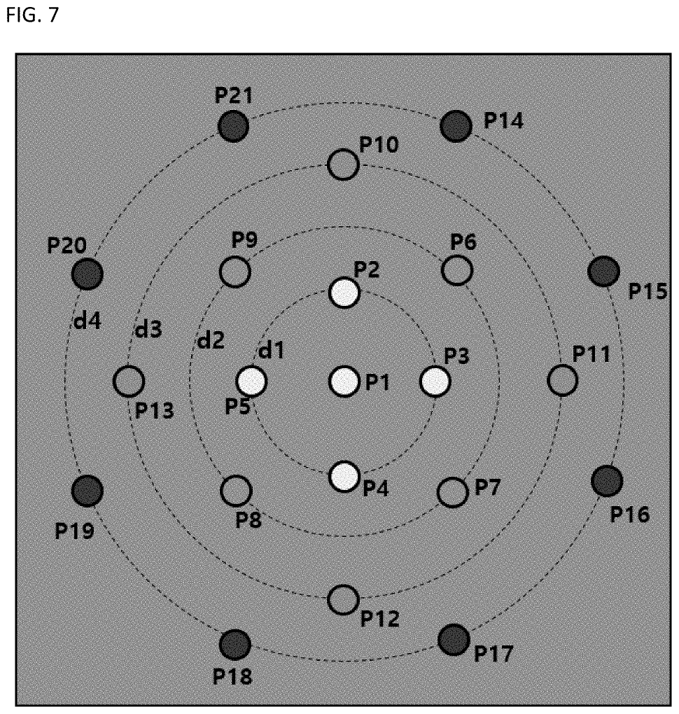

FIGS. 5 to 7 show various embodiments of a microelectrode array pattern according to the present invention.

FIG. 5 is a schematic diagram illustrating a new measurement method for electrodes in an array form proposed in the present invention. In the present invention, a number of electrodes in an array form are used, various biosignals are measured between the number of nearby electrodes, and changes in the biosignals are observed, thereby significantly improving precision and reliability.

According to the present invention, the average of measured values may be obtained for each of the corresponding electrode groups.

In the present invention, it may also be possible to obtain the average of measured values obtained by excluding at least one of an upper limit value and a lower limit value from measured values for each of the corresponding electrode groups.

The array pattern structure of microelectrodes according to the present invention may be implemented in various embodiments.

As an embodiment, there may be possible an embodiment in which a plurality of microelectrodes 210 are arranged in a pattern where the same numbers of microelectrodes are arranged in the lateral and transverse directions thereof and a reference electrode may be any one of the plurality of microelectrodes.

In this embodiment, it is preferable that a plurality of microelectrodes 210 be disposed such that the same odd numbers of microelectrodes are arranged in the lateral and transverse directions (see FIGS. 5 and 6) and a reference electrode be set to a microelectrode located at the center thereof. However, this does not mean that an embodiment in which a plurality of microelectrodes 210 are disposed such that the same even numbers of microelectrodes are arranged in the lateral and transverse directions is excluded from the scope of rights of the present invention.

As another embodiment, there may be possible an embodiment in which a plurality of microelectrodes 210 are arranged in a pattern (not shown) where different numbers of microelectrodes are arranged in the lateral and transverse directions thereof and a reference electrode may be any one of the plurality of microelectrodes.

Meanwhile, the ‘lateral direction’ and the ‘transverse direction’ used herein are not limited to the transversal direction and the horizontal direction, but are based on the concepts also including those arranged in oblique directions.

For example, the microelectrodes may be freely disposed in a vertical direction, a horizontal direction, an oblique direction, or a random direction. However, this embodiment is characterized in that a pattern is formed such that a plurality of other electrodes located at the same distance from a reference electrode are provided.

As an example, FIG. 5 shows a pattern structure in which 25 microelectrodes are disposed in an array form. When a description is given with P13, which is the center one of the above microelectrodes, set as a reference electrode, a total of four microelectrodes P8, P12, P18, and P14 are located at the shortest same distance from an electrode P13.

In the present invention, microelectrodes located at the same distance from a reference electrode are referred to as a corresponding electrode group. Accordingly, the four microelectrodes located at the shortest same distance are referred to as a first corresponding electrode group.

The first corresponding electrode group is present as the four microelectrodes P8, P12, P18, and P14 along lines a1-a2 and a3-a4 in FIG. 5, and there are four measured values with respect to the reference electrode.

In this case, although there is one measured value between two electrodes in a conventional case, four closest electrodes are present in the case of the present invention, and thus there are four measured values.

Four microelectrodes P7, P9, P17, and P19 located at the next shortest same distance from the reference electrode after the first corresponding electrode group constitute a second corresponding electrode group. The four microelectrodes are present on lines b1-b2 and b3-b4 in FIG. 5, and there are four measured values with respect to a reference electrode.

Four microelectrodes P3, P11, P15, and P23 located at the next shorted same distance after the second corresponding electrode group constitute a third corresponding electrode group. The four microelectrodes are present on lines a1-a2 and a3-a4 in FIG. 5, and there are four measured values with respect to a reference electrode.

Eight microelectrodes P6, P20, P2, P24, P4, P22, P10, and P16 located at the next shortest same distance after the third corresponding electrode group constitute a fourth corresponding electrode group. The eight microelectrodes are present on lines c1-c2, c3-c4, c5-c6, and c7-c8 in FIG. 5, and there are eight measured values with respect to the reference electrode.

Four microelectrodes P6, P20, P2, P24, P4, P22, P10, and P16 located at the next shortest same distance after the fourth corresponding electrode group constitute a fifth corresponding electrode group. The four microelectrodes are present on lines b1-b2 and b3-b4 in FIG. 5, and there are four measured values with respect to a reference electrode.

In summary, when an electrode P13 is set as a reference electrode, a total of 24 inter-electrode measured values may be obtained, and thus characteristic values having significantly high precision and reliability may be obtained compared to the conventional measurement method.

FIG. 6 shows an embodiment in which, for example, another electrode position P1 is set as a reference electrode. Also in this embodiment, when corresponding electrode groups from a corresponding electrode group located at the shortest same distance to a corresponding electrode group located at the longest same distance are set and inter-electrode values with the reference electrode are measured, a total of 24 measured values may be obtained.

In this manner, it may be possible to obtain measured values between nearby electrodes at the locations of a total of 25 microelectrodes. When the number of electrodes is 25 as described above, a total of 300 measured values are obtained at one time. Through this, it may be possible to obtain significantly precise and reliable measured values.

When the number of electrodes is n, the number of measured values that can be measured at one time is n(n−1)/2. When the number of electrodes is 100, 4,950 measured values are obtained at one time.

Meanwhile, FIG. 7 shows an embodiment in which a microelectrode array has a concentric pattern structure. As shown in FIG. 7, a plurality of microelectrodes 200 may be disposed at the center of a plurality of concentric circles and on the circumferences of the respective concentric circles, a reference electrode may a microelectrode disposed at the center of the circles, and each corresponding electrode group may include microelectrodes arranged on the circumference of a corresponding one of the concentric circles.

According to the present invention, as proposed by the above various embodiments, a distribution of measured values may be represented by a number of measured values, and significantly precise measurement may be possible.

In the present invention, the driving power unit 400 may be any one of a coin-type battery, a film-type thin film battery, a piezoelectric-type rechargeable battery, a triboelectric-type rechargeable battery, a solar-type wireless power transmission unit, an RF wireless power transmission unit, and a biofuel cell.

In the present invention, in order to minimize the size of the device, a micro-sized coin-type battery or a film-type thin-film battery may be used. In addition, for instantaneous high energy storage, it may also be possible to use a capacitor in the form of a super-capacitor instead of a general battery.

In the present invention, the wireless communication unit 500 may use any one of a Bluetooth communication device, a Wi-Fi communication device, and a BCC communication device.

For wireless communication, it may be configured as an RF communication device such as a Bluetooth communication device or a Wi-Fi communication device.

When the wireless communication module is implanted into a living body, a problem may arise in that RF performance is attenuated in the living body. Accordingly, in order to prevent such attenuation, it may also be possible to construct a body channel communication (BCC) device using a living body and transmit or receive signals via BCC communication. In addition, wireless communication using various types of wireless communication devices may also be possible.

The embodiments described herein and the accompanying drawings are merely illustrative of part of the technical spirit included in the present invention. Accordingly, it is obvious that the embodiments disclosed in the present specification are not intended to limit the technical spirit of the present disclosure, but are intended to describe the technical spirit, so that the scope of the technical spirit of the present invention is not limited by these embodiments. Modifications and specific embodiments that may be easily inferred by those skilled in the art without departing from the scope of the technical spirit included in the specification and drawings of the present invention should be interpreted as being included in the scope of the present invention.

1. A biosignal measuring and stimulating device having bioelectrodes in which a signal measurement unit including bioelectrodes composed of a plurality of microelectrodes is disposed on a substrate, wherein the signal measurement unit, a measured signal processing unit, and at least one of a driving power unit and a wireless communication unit are disposed on the substrate in a vertical or lateral direction, and the biosignal measuring and stimulating device measures biosignals or stimulates from the microelectrodes formed in an array pattern.

2. The biosignal measuring and stimulating device of claim 1, wherein the microelectrodes are provided as solder bumps.

3. The biosignal measuring and stimulating device of claim 2, wherein the solder bumps have a round shape or a tapered cone shape widening downward.

4. The biosignal measuring and stimulating device of claim 1, wherein the driving power unit is any one of a coin-type battery, a film-type thin film battery, a piezoelectric-type rechargeable battery, a triboelectric-type rechargeable battery, a solar-type wireless power transmission unit, an RF wireless power transmission unit, a biofuel cell, and a super-capacitor.

5. The biosignal measuring and stimulating device of claim 1, wherein the wireless communication unit uses any one of a Bluetooth communication device, a Wi-Fi communication device, and a BCC communication device.

6. The biosignal measuring and stimulating device of claim 1, wherein the microelectrodes formed in the array pattern are set as one microelectrode, which is a reference electrode, and a plurality of corresponding electrode groups each including other microelectrodes located at a same distance from the reference electrode, and, for each of the corresponding electrode groups, an average of measured values of biosignals between the reference electrode and the micro-electrodes of the corresponding electrode group is obtained.

7. The biosignal measuring and stimulating device of claim 6, wherein an average of measured values obtained by excluding at least one of an upper limit value and a lower limit value from measured values for each of the corresponding electrode groups is obtained.

8. The biosignal measuring and stimulating device of claim 1, wherein the plurality of microelectrodes are arranged in a pattern where same numbers of microelectrodes are arranged in lateral and transverse directions of the pattern, and a reference electrode is any one of the plurality of microelectrodes.

9. The biosignal measuring and stimulating device of claim 8, wherein the plurality of microelectrodes are arranged such that same odd numbers of microelectrodes are arranged in lateral and transverse directions of the pattern, and the reference electrode is set to a microelectrode located at a center of the pattern.

10. The biosignal measuring and stimulating device of claim 1, wherein the plurality of microelectrodes are arranged in a pattern where different numbers of microelectrodes are arranged in lateral and transverse directions of the pattern, and a reference electrode is any one of the plurality of microelectrodes.

11. The biosignal measuring and stimulating device of claim 1, wherein the plurality of microelectrodes are disposed at a center of a plurality of concentric circles and on circumferences of the respective concentric circles, a reference electrode is a microelectrode disposed at the center of the circles, and each corresponding electrode group includes microelectrodes arranged on a circumference of a corresponding one of the concentric circles.
